(19) United States
(12) Patent Application Publication
(10) Pub. No.: US 2013/0022995 A1
(43) Pub. Date: Jan. 24, 2013

(54) METAL NANOWIRE INCLUDING GOLD NANOCLUSTERS ON A SURFACE THEREOF FOR BINDING TARGET MATERIAL AND METHOD OF BINDING THE TARGET MATERIAL TO THE METAL NANOWIRE

(71) Applicants: Gyeong-su PARK, Suwon-si (KR); Min-seok KIM, Yongin-si (KR); Seong-yong PARK, Seongnam-si (KR); Jeong-gun LEE, Seoul (KR); Young-nam KWON, Gunpo-si (KR); Yeon-jeong KIM, Yongin-si (KR)

(72) Inventors: Gyeong-su PARK, Suwon-si (KR); Min-seok KIM, Yongin-si (KR); Seong-yong PARK, Seongnam-si (KR); Jeong-gun LEE, Seoul (KR); Young-nam KWON, Gunpo-si (KR); Yeon-jeong KIM, Yongin-si (KR)

(73) Assignee: SAMSUNG ELECTRONICS CO., LTD., Suwon-si (KR)

(21) Appl. No.: 13/550,823

(22) Filed: Jul. 17, 2012

(30) Foreign Application Priority Data
Jul. 18, 2011 (KR) 10-2011-0071090

(51) Int. Cl. B32B 15/01; G01N 33/574; C23C 16/06; B82Y 40/00; B82Y 30/00

(52) U.S. Cl. 435/7.23; 428/655; 427/250; 977/762; 977/773; 977/890

(57) ABSTRACT

A metal nanowire including gold nanoclusters on the surface thereof for binding a target material and a method of binding the target material to the metal nanowire are provided.

### CROSS-REFERENCE TO RELATED APPLICATIONS

This application claims priority to Korean Patent Application No. 10-2011-0071090, filed on Jul. 18, 2011, and all the benefits accruing therefrom under 35 U.S.C. §119, the content of which in its entirety is herein incorporated by reference.

### BACKGROUND

1. Field

The present disclosure relates to a metal nanowire including gold nanoclusters on the surface thereof for binding a target material and a method of binding the target material to the metal nanowire.

2. Description of the Related Art

A nanowire is a nanostructure, with a diameter in the order of nanometers. Alternatively, nanowires can be defined as structures that have a thickness or diameter in the order of nanometers, for example, constrained to tens of nanometers or less and an unconstrained length. Many different types of nanowires exist.

Techniques for creating a nanowire typically include suspension, vapor-liquid-solid (“VLS”) synthesis, and solution-phase synthesis methods. A suspended nanowire may be produced by chemical etching, or bombardment, typically with highly energetic ions, of a larger wire. The solution-phase synthesis method allows a nanowire to grow in solution.

Nanowires may be synthesized using the VLS synthesis method. The VLS synthesis method typically uses laser ablated particles or a feed gas (such as silane) as a source material. The source material is then exposed to a catalyst. For nanowires, the catalyst may be liquid metal nanoclusters, which may be purchased in colloidal form and deposited on a substrate or self-assembled from a thin film by dewetting. This process may produce crystalline nanowires in the case of semiconductor materials.

The source material enters these nanoclusters and begins to saturate the nanocluster. Once supersaturated, the source material solidifies and grows outward from the nanocluster. A length of the final product may be adjusted by stopping providing of the source material. Compound nanowires with super-lattices of alternating materials may be created by switching source materials while still in the growth phase.

Semiconductor nanowires have received much attention due to unique properties thereof. A silicon nanowire may be synthesized using the VLS synthesis method. Heavy metals such as gold (Au), silver (Ag), cobalt (Co), copper (Cu), nickel (Ni) and titanium (Ti) may be used as a catalyst.

### SUMMARY

Provided is a metal nanowire including gold nanoclusters on a surface thereof for binding a target material to the metal nanowire with improved efficiency.

Provided is a method of binding the target material to the metal nanowire including gold nanoclusters on a surface thereof with improved efficiency.

Additional features will be set forth in part in the description which follows and, in part, will be apparent from the description, or may be learned by practice of the embodiments described herein.

In an embodiment, a metal nanowire includes gold nanoclusters on a surface thereof, where the gold nanoclusters bind a target material to the metal nanowire.

In another embodiment, a method of preparing a metal nanowire including gold nanoclusters on a surface thereof includes providing a gold thin layer on a metal substrate, forming a gold-metal island by performing a first calcination process on the metal substrate including the gold thin layer thereon in a chamber in a hydrogen atmosphere, and performing a second calcination process on the metal substrate including the gold-metal island while injecting a mixed gas into the chamber to grow the metal nanowire including gold nanoclusters on the surface thereof.

In another embodiment, a method of binding a target material in a sample to a metal nanowire including gold nanoclusters on a surface thereof includes binding the target material to the metal nanowire by contacting the metal nanowire including the gold nanoclusters on the surface thereof to the sample including the target material.

### BRIEF DESCRIPTION OF THE DRAWINGS

These and/or other features will become apparent and more readily appreciated from the following description of the embodiments, taken in conjunction with the accompanying drawings of which:

FIG. 1 is a scanning electron microscope (“SEM”) image of an embodiment of a silicon nanowire including gold nanoclusters on a surface thereof at a high density according to the invention;

FIG. 2A is an enlarged view of the silicon nanowire of FIG. 1 and including the gold nanoclusters on the surface thereof at a high density;

FIG. 2B is a partial enlarged view of an upper part of the silicon nanowire of FIG. 1, and including the gold nanoclusters on the surface thereof at a high density;

FIG. 2C is a partial enlarged view of a central part of the silicon nanowire of FIG. 1 and including the gold nanoclusters on the surface thereof at a high density;

FIG. 3A shows a transmission electron microscopy (“TEM”) image of a cross-section of the silicon nanowire of FIG. 1 and including the gold nanoclusters on the surface thereof at a high density;

FIG. 3B shows a Z-contrast scanning transmission electron microscopy (“STEM”) image of a cross-section of the silicon nanowire of FIG. 1 and including the gold nanoclusters on the surface thereof at a high density;

FIG. 4A shows a high-resolution Z-contrast image of the gold nanoclusters included in the silicon nanowire of FIG. 1;

FIGS. 4B, 4C and 4D are graphs showing Si and gold atoms distinguished by differences in contrast intensities of a, b and c regions of FIG. 4A, respectively;

FIG. 5 is a graph showing monochrome-electron energy loss spectroscopy (“EELS”) data obtained by measuring and comparing surface plasmon excitation energies of a gold cap and a gold nanocluster in an embodiment of the silicon nanowire of FIG. 1;

FIG. 6A is an enlarged view of a surface of an embodiment of a silicon nanowire including the gold nanoclusters on the surface thereof at a high density obtained by performing a thermal treatment on the nanowire of FIG. 1 at a temperature of 700° C.;

FIG. 6B is a graph showing a distribution of the gold nanoclusters of FIG. 6A according to the size thereof;

FIG. 7A is an enlarged view of a surface of an embodiment of the silicon nanowire including the gold nanoclusters on the surface thereof at a high density obtained by performing a thermal treatment on the nanowire of FIG. 1 at a temperature of 800° C.;

FIG. 7B is a graph showing a distribution of the gold nanoclusters of FIG. 7A according to the size thereof;

FIG. 8A is an enlarged view of a surface of an embodiment of the silicon nanowire including the gold nanoclusters on the surface thereof at a high density obtained by performing a thermal treatment on the nanowire of FIG. 1 at a temperature of 900° C.;

FIG. 8B is a graph showing a distribution of the gold nanoclusters of FIG. 8A according to the size thereof;

FIG. 9 is a graph showing a number of cells attached to gold nanowires or a gold plate after about 5 minutes of incubation according to each of the gold nanowires and the gold plate;

FIG. 10 is a graph showing a number of cells attached to gold nanowires or a gold plate according to incubation time and according to each of the gold nanowires and the gold plate; and

FIG. 11 is an image showing a number of cells attached to the gold nanoclusters according to incubation time.

### DETAILED DESCRIPTION

The invention now will be described more fully hereinafter with reference to the accompanying drawings, in which various embodiments are shown. This invention may, however, be embodied in many different forms, and should not be construed as limited to the embodiments set forth herein. Rather, these embodiments are provided so that this disclosure will be thorough and complete, and will fully convey the scope of the invention to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

The terminology used herein is for the purpose of describing particular embodiments only and is not intended to be limiting. As used herein, the singular forms “a,” “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

An embodiment of the invention is directed to a metal nanowire including gold nanoclusters provided on a surface thereof.

In an embodiment, the metal nanowire including gold nanoclusters on the surface thereof may exist in a form of a composition.

In such an embodiment, the composition may include a pharmaceutically acceptable carrier, diluent or excipient. The carrier, diluent or excipient may be any carrier, diluent or excipient well known in the art.

In an embodiment, the metal of the metal nanowire may include at least one metal selected from the group consisting of a transition metal, a lanthanide, a group 13 element (except boron), and a group 14 element (except carbon and silicon). In an embodiment, the metal may include at least one metal selected from the group consisting of silicon (Si), nickel (Ni), iron (Fe), silver (Ag), aluminum (Al), germanium (Ge), gadolinium (Gd), copper (Cu), indium (In), titanium (Ti) and lead (Pb). In one embodiment, for example, the metal may be silicon.

In an embodiment, a cross-section of the nanowire may have an arbitrary shape. In one embodiment, for example, the cross-section may be a circle, a triangle, a pentagon, a hexagon or any combination thereof. In an embodiment, the metal nanowires may have a same shape with each other. In another embodiment, the metal nanowires may have cross-section shapes from different from each other. In an embodiment, an end portion of the nanowire may include a gold cap having a hemispherical shape.

In an embodiment, the nanowire may have a diameter in a range from about 10 nanometers (nm) to about 50 nanometers (nm). In such an embodiment, the nanowire may have a diameter in a range from, for example, about 10 nm to about 40 nm, about 10 nm to about 30 nm, about 10 nm to about 20 nm, about 10 nm to about 15 nm, about 15 nm to about 40 nm, about 15 nm to about 30 nm, about 15 nm to about 20 nm, about 20 nm to about 40 nm, or about 20 nm to about 30 nm. In an embodiment, a length of the nanowire may be in a range from about 0.5 to about 20 μm. In such an embodiment, the length of the nanowire may be in a range from, for example, about 0.5 micrometer (μm) to about 15 micrometers (μm), about 0.5 μm to about 10 μm, about 0.5 μm to about 7 μm, about 0.5 μm to about 5 μm, about 1 μm to about 15 μm, about 1 μm to about 10 μm, about 1 μm to about 7 μm, about 1 μm to about 5 μm, about 3 μm to about 15 μm, about 3 μm to about 10 μm, about 3 μm to about 7 μm, or about 3 μm to about 5 μm. The diameter of the metal nanowire may be measured via a cross-section that is cut substantially perpendicular with respect to the length direction of the metal nanowire. In an embodiment, where the metal nanowire has a polygonal, for example hexagonal shape cross-section, the cross-section may be measured as a length of a major axis (a line connecting opposite vertices) of the polygonal, for example hexagonal shape. The diameter and length of the metal nanowire such as silicon nanowire may be adjusted based on conditions of a manufacturing process.

In an embodiment, the nanowires may have a disengaged form, an immobilized form on a substrate, an immobilized form on an interior wall of a container or a channel, or a combination thereof. In an embodiment, the nanowires in the disengaged form may be mixed with a sample in a container. In an embodiment, where the nanowires have the immobilized form on a substrate or in the immobilized form on the inner wall of a container, the substrate and the container or the channel may include at least one of glass, plastic, metal and a combination thereof. In such an embodiment, the plastic may be one of polyethylene, polypropylene, polystyrene, polyvinylchloride and a combination thereof. In such an embodiment, the metal may be at least one of silicon (Si), nickel (Ni), iron (Fe), silver (Ag), aluminum (Al), germanium (Ge), gadolium (Gd), copper (Cu), indium (In) and lead (Pb). In such an embodiment, the substrate may have a flat or irregular surface. In such an embodiment, the channel may be a microchannel or a nanochannel. For the term “microchannel” or “nanochannel,” the microchannel may be defined as a channel having a cross-sectional length in a range from about 1 μm to about 1,000 μm, and the nanochannel may be defined as a channel having a cross-sectional length in a range from about 1 nm to about 1,000 nm. In such an embodiment, the immobilization may be done by immobilizing disengaged nanowires on the interior wall of the substrate and the container or the channel separately with the synthesis of the nanowire, or by growing nanowires thereon. In an embodiment, the metal nanowires may be synthesized using a vapor-liquid-solid (“VLS”) synthesis method.

In an embodiment, “gold nanoclusters” indicates gold agglomerated in the form of nanodots. In an embodiment, the gold nanoclusters may have a circular shape or an irregular shape. In an embodiment, the gold nanoclusters may have a size less than a diameter of the metal nanowire, for example, a size less than about 500 nm, which is characteristic of a nanowire, for example, about 1 nm to about 100 nm in terms of an average cross-sectional length. In an embodiment, the gold nanoclusters may have an average cross-sectional length of about 1 nm to about 10 nm. In such an embodiment, the gold nanoclusters may have an average cross-sectional length of, for example, about 1 to about 7 nm, about 1 to about 5 nm, about 1 to about 3 nm, about 2 to about 10 nm, about 2 to about 7 nm, about 2 to about 5 nm, about 3 to about 10 nm, about 3 to about 7 nm, or about 3 to about 5 nm. In an embodiment, where the metal nanoclusters have a circular shape, the size of the metal nanoclusters may be defined as a diameter of the metal nanoclusters. In an embodiment, where the metal nanoclusters have an irregular shape, the size of the metal nanoclusters may be defined as a length of a major axis of the metal nanoclusters.

In an embodiment, the gold nanoclusters may be provided on a surface of metal nanowire at a high density. In an embodiment, the gold nanoclusters may have a density of about 1×10⁶ nanoclusters per square centimeter (nanoclusters/cm²) to about 1×10¹⁶ nanoclusters per square centimeter (nanoclusters/cm²). In such an embodiment, the gold nanoclusters may has a density of, for example, about 1×10⁷ nanoclusters/cm² to about 1×10¹⁶ nanoclusters/cm², about 1×10⁹ nanoclusters/cm² to about 1×10¹⁶ nanoclusters/cm², about 1×10¹¹ nanoclusters/cm² to about 1×10¹⁶ nanoclusters/cm², about 1×10¹³ nanoclusters/cm² to about 1×10¹⁶ nanoclusters/cm², about 1×10¹⁴ nanoclusters/cm² to about 1×10¹⁶ nanoclusters/cm², about 1×10⁷ nanoclusters/cm² to about 1×10¹⁶ nanoclusters/cm², about 1×10⁹ nanoclusters/cm² to about 1×10¹⁴ nanoclusters/cm², about 1×10¹¹ nanoclusters/cm² to about 1×10¹³ nanoclusters/cm², about 1×10¹³ nanoclusters/cm² to about 1×10¹⁶ nanoclusters/cm², or about 1×10¹⁴ nanoclusters/cm² to about 1×10¹⁵ nanoclusters/cm². In such an embodiment, the high density of gold nanoclusters may be uniformly arranged, and the gold nanoclusters may be arranged with intervals of about 1 nm to about 100 nm.

The range of distribution, interval of arrangement (e.g., spacing among the metal nanoclusters) and size of the metal nanoclusters may vary according to conditions of a manufacturing process, and may be adjusted according to the purpose of use.

In an embodiment, the gold nanoclusters may be unmodified gold nanoclusters or modified gold nanoclusters. The term “unmodified gold nanocluster” indicates that the surface of a gold nanocluster is not bound or blocked by any other chemical substance. In an exemplary embodiment, where the gold nanoclusters are unmodified gold nanoclusters, the reactivity of gold existing on the surface of the gold nanocluster remains substantially the same. In such an embodiment, where the gold nanocluster is an unmodified gold cluster, a target material may be a material having a thiol group. In one embodiment, for example, the target material may be a protein including an amino acid residue having a thiol group in it or a cell including the protein.

The term “modified gold nanocluster” indicates that the surface of the gold nanocluster is bound or blocked by another chemical substance. In an embodiment, where the gold nanoclusters are the modified gold nanoclusters, the surface of the gold nanoclusters are bound or blocked by a chemical substance, which may be a substance binding specifically or nonspecifically to a target material. In one embodiment, for example, the chemical substance may be an antibody when a target material is an antigen, an antigen when a target material is an antibody, a receptor when a target material is a ligand, or an inhibitor or an activator of an enzyme when a target material is an enzyme. In such an embodiment, the chemical substance may be, for example, an antibody binding to a specific antigen. In such an embodiment, modifying the surface of gold may be performed using a conventional method, well known in the art. In one embodiment, for example, an antigen or an antibody having a thiol group may be immobilized on the surface of gold by using the affinity between gold and sulfur.

In an embodiment, the metal nanowire with the gold nanoclusters may include a silicon material. In such an embodiment, the metal nanowire with the gold nanoclusters may include, for example, amorphous silicon, crystalline silicon, silicon comprising silica or any silicon material, regardless of the shape and size thereof. In an embodiment, the silicon nanowire with the gold nanoclusters may have circular shaped cross-section or a polygonal-shaped cross-section (e.g., a hexagonal-shaped cross-section). In an embodiment, an upper end portion of the silicon nanowire may include a metal cap having a hemispherical shape.

The metal nanowires may be immobilized on a substrate at a high density. The metal nanowires immobilized on the substrate may have a density of about 1×10³ nanoclusters/cm² or more. In an embodiment, the metal nanowires immobilized on the substrate may have a density of, for example, about 1×10⁴ nanoclusters/cm² or more, about 1×10⁵ nanoclusters/cm² or more, or about 1×10⁷ nanoclusters/cm² or more. In an embodiment, the metal nanowires may have a density in a range of about 1×10³ nanoclusters/cm² to about 1×10¹⁶ nanoclusters/cm² on the substrate. In such an embodiment, the metal nanowires may have a density in a range of, for example, about 1×10⁴ nanoclusters/cm² to about 1×10¹⁶ nanoclusters/cm², about 1×10⁵ nanoclusters/cm² to about 1×10¹⁶ nanoclusters/cm², about 1×10⁶ nanoclusters/cm² to about 1×10¹⁶ nanoclusters/cm², about 1×10⁷ nanoclusters/cm² to about 1×10¹⁶ nanoclusters/cm², about 1×10⁹ nanoclusters/cm² to about 1×10¹⁶ nanoclusters/cm², about 1×10¹¹ nanoclusters/cm² to about 1×10¹⁶ nanoclusters/cm², about 1×10¹³ nanoclusters/cm² to about 1×10¹⁶ nanoclusters/cm², about 1×10¹⁴ nanoclusters/cm² to about 1×10¹⁶ nanoclusters/cm², about 1×10⁷ nanoclusters/cm² to about 1×10¹⁵ nanoclusters/cm², about 1×10⁹ nanoclusters/cm² to about 1×10¹⁴ nanoclusters/cm², about 1×10¹¹ nanoclusters/cm² to about 1×10¹³ nanoclusters/cm², about 1×10¹³ nanoclusters/cm² to about 1×10¹⁵ nanoclusters/cm², or about 1×10¹⁴ nanoclusters/cm² to about 1×10¹⁵ nanoclusters/cm².

In an embodiment, the metal nanowires may be uniformly or non-uniformly arranged on the substrate. In an embodiment, the metal nanowires may be arranged in a patternized form. In an embodiment, each metal nanowire group may be arranged with an interval in a range of, for example, about 1 nm to about 100 nm. In an embodiment, the metal nanowires may be vertically immobilized on the substrate. In such an embodiment, an end of the metal nanowire is attached to the substrate and another end is exposed to the space facing the substrate, such that the nanowire may be in contact with a target material. In such an embodiment, a distal end of the metal nanowire relative to the substrate is exposed and not immobilized on the substrate, and the distal end of the metal nanowire may have a tip substantially surrounded by gold nanoclusters in a form of a cap. In an embodiment, a distal portion of the metal nanowire relative to the substrate is exposed and not immobilized on the substrate, and the distal portion of the metal nanowire may have gold nanoclusters on a surface thereof. In such an embodiment, the substrate may include a metal, which may be the same or different as the metal of the metal nanowire. In one embodiment, for example, the metal nanowire is a silicon nanowire, and the substrate may be a silicon substrate.

The target material may include a material derived from an organism or a part thereof. In an embodiment, the target material may be a cell, a virus or a tissue, for example. In an embodiment, the target material may be a biomolecule. In such an embodiment, the biomolecule may be, for example, a nucleic acid, a protein, a sugar or an amino acid, for example. In an embodiment, the biomolecule may be, for example, a material having a thiol group. In such an embodiment, the material having a thiol group may be, for example, a protein, a cell or a tissue including a cysteine residue. In an embodiment, the target material may be an antigen existing on a surface of a cell or a material including the antigen. In such an embodiment, the target material may be a marker antigen to a specific cancer, such as an epithelial cell adhesion molecule (“EpCAM”). In an embodiment, the target material may be a circulating tumor cell (“CTC”). In such an embodiment, the CTC content in a medium may be about 10⁻⁷ cells per milliliter (cells/ml) to about 10⁻⁹ cells per milliliter (cells/ml), where the medium may be body fluid such as blood, serum, urine or saliva, or may be liquid medium such as a buffer or water.

The metal nanowire may be prepared by a method including providing a gold thin film layer on a metal substrate; forming a gold-metal island by performing a first calcination process on the metal substrate including the gold thin film layer in a chamber in a hydrogen atmosphere; and growing the metal nanowire including gold nanoclusters on the surface thereof by performing a second calcination process on the metal substrate including the gold-metal island while injecting a mixed gas into the chamber. The chamber may be a chemical vapor deposition (“CVD”) chamber.

In the providing the gold thin film layer on the metal substrate, the metal substrate may include at least one of silicon (Si), nickel (Ni), iron (Fe), silver (Ag), aluminum (Al), germanium (Ge), gadolium (Gd), copper (Cu), indium (In) and lead (Pb).

In an embodiment, the gold thin film layer may have a thickness in a range of about 1 nm to about 100 nm. In such an embodiment, the gold thin film layer may have a thickness of, for example, in a range from about 1 nm to about 10 nm, using a method, for example, sputtering, CVD, spin coating, atomic layer deposition (“ALD”), or metal organic chemical vapor deposition (“MOCVD”). In an embodiment, the gold thin film layer may be formed on at least one surface of the metal substrate. In one embodiment, for example, the gold thin film layer may be formed on both surfaces of the metal substrate.

In an embodiment, the gold thin film layer is formed on the metal substrate, and then the metal nanowire is grown. In such an embodiment, the metal nanowire may be grown using a method, for example, rapid thermal chemical vapor deposition (“RTCVD”), laser thermal chemical vapor deposition (“LTCVD”) or MOCVD.

In an embodiment, the metal substrate, for example, a silicon substrate including the gold thin film layer, may be positioned inside a chamber, for example, a CVD chamber, to grow the metal nanowire. In such an embodiment, the chamber may be, for example, a CVD chamber, using a halogen lamp or laser.

In an embodiment, where the first calcination process is performed in the chamber, for example a CVD chamber, the gold and the metal (e.g., a silicon substrate) react with each other, and thus the gold-metal (e.g., gold-silicon) island is substantially uniformly formed on the substrate. The gold-metal (e.g., gold-silicon) island, which is nano-sized, may be a silicide-type particulate material.

The first calcination process may be performed in a hydrogen atmosphere, and may be performed in a vacuum atmosphere at, for example, a pressure in a range of about 0.1 torr (Torr) to about 500 torrs (Torr). The first calcination process may be performed at a temperature in a range of about 300° C. to about 1,000° C. during a time period in a range of about 5 minutes to about one hour.

After the gold-metal (for example, gold-silicon) island is uniformly formed on the silicon substrate by the first calcination process, the second calcination process is performed to grow the metal nanowire with the gold nanoclusters.

In an embodiment, the second calcination process may be performed when the chamber is maintained at a pressure in a range of about 0.1 Torr to about 10 Torr and at a temperature in a range of about 500° C. to about 600° C., while injecting a mixed gas into the chamber, during a time period in a range of about 0.1 to about 10 hours. In an embodiment, the mixed gas may be a mixture of SiH₄ and H₂, for example, but not being limited thereto. In such an embodiment, the amount of SiH₄ may be in a range from about 1 sccm (standard cubic centimeters per minute) to about 10 sccm (standard cubic centimeters per minute), and the amount of H₂ may be in a range from about 10 sccm to about 100 sccm.

In an embodiment, after the second calcination process is performed, the metal nanowire with the gold nanoclusters uniformly on the surface thereof at a high density may be obtained.

In an embodiment, the size, degree of distribution, interval of arrangement e.g., spacing among the nanoclusters of the metal nanoclusters or the diameter and length of the silicon nanowire may be controlled by adjusting, for example, the pressure, temperature or retention time within the chamber during the first and second calcination processes. In one embodiment, for example, properties of the metal nanowire including gold nanoclusters may be controlled by adjusting the pressure, temperature or retention time of the CVD chamber.

After the metal nanowire with the gold nanoclusters on the surface thereof is prepared, a thermal treatment may be additionally performed on the metal nanowire at a temperature in a range of about 300° C. to about 1,000° C. during a time period in a range of 0.1 hour to 10 hours such that the size or density of the gold nanoclusters are adjusted.

In such an embodiment, the metal nanowire with the gold nanoclusters on the surface thereof at a high density may have an improved electron capture characteristic, improved electrical conductivity, and an improved optical characteristic including light absorption or optical emitting properties, and thus the metal nanowire may be used in various electric devices.

According to another embodiment of the invention, a method of binding a target material in a sample to a metal nanowire includes contacting the metal nanowire including gold nanoclusters on the surface thereof to the sample including the target material to bind the target material to the metal nanowire.

In such an embodiment, the contacting the metal nanowire to the sample may include mixing the metal nanowire and the sample and allowing the mixture of the metal nanowire and the sample to stand or stirring the mixture of the metal nanowire and the sample. The stirring the mixture may be performed using a conventional method. The stirring the mixture may be, for example, at least one of rotating, vortexing, rotating magnetic beads and inverting. In an embodiment, the contacting the metal nanowire to the sample may include flowing the sample while contacting the metal nanowire.

In an embodiment, the contacting the metal nanowire to the sample may be performed in a liquid medium. The liquid medium may be selected according to a target material and a sample. In an embodiment, the liquid medium may be, for example, water, a buffer or a diluent. In such an embodiment, where the liquid medium is a buffer, the buffer may be, for example, a phosphate buffered saline (“PBS”) or Tris buffer.

The sample may include the target material at a very low concentration. The sample may include a CTC with a concentration in a range of about 10⁻⁷ cells/ml through about 10⁻⁹ cells/ml. The sample may include body fluid derived from an organism such as blood, serum, urine and saliva, for example.

In an embodiment, the method may further include separating a target material from the nanowire. In such an embodiment, the separating may be performed using a conventional method that breaks the bound target material away from the nanowire. In an embodiment, an eluent may be used to break the bond between the nanowire and the target material. In another embodiment, the bond may be broken by controlling properties such as pH, salt concentration and conductivity of the mixture of the target material and the nanowire. In such an embodiment, the bond between the nanowire and the target material includes covalent or non-covalent bond.

In an embodiment, the method may further include washing the nanowire after the binding. In such an embodiment, materials non-specifically bound to the nanowire other than the target material may be removed by the washing the nanowire. The washing may be performed using, for example, a buffer (e.g., PBS or Tris) or water. In such an embodiment, a washing buffer may be selected according to a target material and a nanowire.

In an embodiment, the method may further include lysing the target material by irradiating a laser on the nanowire after the binding. In such an embodiment, the laser may be a pulse laser or a continuous wave laser. The pulse laser may have an output of about 1 millijoule per pulse (mJ/pulse). The continuous wave laser may have an output of about 10 milliwatts (mW) or more. The light produced by the laser may have a wavelength of about 400 nanometers (nm) or more. In an embodiment including irradiating the laser on the nanowire, a temperature may increase due to the interaction between the laser light and the gold clusters, and the temperature of the mixture including the nanowire and the target material may also increase such that the target material may be lysed. In an embodiment, the target material may be a cell. In such an embodiment, the cell may be an animal cell, a plant cell, a bacterium or a virus, for example.

The contacting may be performed in vitro or in vivo. In an embodiment, for example, the contacting may be performed in the body of a mammal, for example, a human, a dog, a pig, a cow or a sheep. According to an exemplary embodiment of the method described herein, a binding efficiency with respect to a cell is substantially high such that the metal nanowire may be bound to the target material existing at a very low concentration. The bound target material may be used for detection or may be destroyed and removed. In an embodiment, the removing of the target material includes physically removing or removing an activity thereof. The activity of the target material may be, for example, proliferation of a cell or a virus. As described herein, after binding the target material and the nanowire, the metal nanowire or an area substantially surrounding the metal nanowire, for example, surrounding area very near the metal nanowire (e.g., in a range about 10 nm to about 1 millimeter (mm)), may be heated by heating the metal nanowire including gold nanoclusters on the surface thereof by irradiating the laser. Thus, the target material may be removed by heating only the region where the target material is binding, thus minimizing harm to an organism including the target material.

In an embodiment, the metal nanowire may be prepared using a method including providing a gold thin film layer on a metal substrate; forming a gold-metal island by performing a first calcination process on a metal substrate including the gold thin film layer in a chamber in a hydrogen atmosphere; and growing the metal nanowire including gold nanoclusters on the surface thereof by performing a second calcination process on the silicon substrate including the gold-metal island while injecting a mixed gas into the chamber. In such an embodiment, the chamber may be a CVD chamber.

The metal used in the providing the gold thin film layer on the metal substrate may be at least one of a transition metal, a lanthanide, a group 13 element (except boron) and a group 14 element (except carbon and silicon). In such an embodiment, the metal may include at least one of silicon (Si), nickel (Ni), iron (Fe), silver (Ag), aluminium (Al), germanium (Ge), gadolinium (Gd), copper (Cu), indium (In), titanium (Ti) and lead (Pb), for example.

In an embodiment, the gold thin film layer provided on the metal substrate may have a thickness in a range from about 1 nm to about 100 nm. In such an embodiment, the gold thin film may have a thickness in a range, for example, from about 1 nm to about 10 nm, using a method, for example, sputtering, CVD, spin coating, ALD or MOCVD. In an embodiment, the gold thin film layer may be formed on at least one surface of the metal substrate. In an alternative embodiment, the gold thin film may be formed on both surfaces of the metal substrate.

The gold thin film layer is formed on the metal substrate, and then the metal nanowire is grown. The metal nanowire may be grown using a method, for example, RTCVD, LTCVD or MOCVD.

In an embodiment, the metal substrate (e.g., a silicon substrate) including the gold thin film layer may be positioned inside the chamber such as a CVD chamber to grow the metal nanowire. In such an embodiment, the chamber may be a CVD chamber using a halogen lamp or laser.

When the first calcination process is performed in the chamber, the gold and the metal substrate (e.g., silicon substrate) react with each other, and thus the gold-metal (e.g., gold-silicon) island is uniformly formed on the substrate. The gold-metal (for e.g., gold-silicon) island, which is nano-sized, may be a silicide-type particulate material.

The first calcination process may be performed in a hydrogen atmosphere, and may be performed in a vacuum atmosphere, for example, at a pressure in a range from about 0.1 Torr to about 500 Torr. The first calcination process may be performed at a temperature in a range of about 300° C. to about 1,000° C. for about 5 minutes to about one hour.

After the gold-metal (e.g., gold-silicon) island is uniformly formed on the silicon substrate by the first calcination process, the second calcination process is performed to grow the metal nanowire with the gold nanoclusters.

The second calcination process may be performed when the chamber is maintained at a pressure in a range from about 0.1 Torr to about 10 Torr and at a temperature of about 500° C. to about 600° C., while injecting a mixed gas into the chamber, for about 0.1 hour to about 10 hours. In an embodiment, the mixed gas may be a mixture of SiH₄ and H₂. In such an embodiment, the amount of SiH₄ may be in a range from about 1 sccm to about 10 sccm, and the amount of H₂ may be in a range from about 10 sccm to about 100 sccm.

After the second calcination process is performed, the metal nanowire including the gold nanoclusters uniformly formed on the surface thereof at a high density may be obtained.

The size, degree of distribution, interval of arrangement (e.g., spacing among the nanoclusters of the metal nanoclusters) or the diameter and length of the silicon nanowire may be controlled by adjusting, for example, the pressure, temperature or retention time within the chamber during the first and second calcination processes. In an embodiment, for example, the properties of the metal nanowire including gold nanoclusters may be controlled by adjusting the pressure, temperature, retention time within the chamber.

After the metal nanowire with the gold nanoclusters formed on the surface thereof is prepared, a thermal treatment may be additionally performed on the metal nanowire (hereinafter, calls “third calcination”). The third calcination may be performed at a temperature in a range of, for example, about 300° C. to about 1,000° C., about 500° C. to about 1,000° C., about 700° C. to about 1,000° C., about 800° C. to about 1,000° C., about 300° C. to about 900° C., about 300° C. to about 800° C., about 300° C. to about 700° C., about 300° C. to about 500° C., about 300° C. to about 400° C., or about 700° C. to about 900° C. The time for the third calcination may be appropriately selected, which is well known in the art. In an embodiment, the heat treatment may be performed during a time period in a range of about 0.1 hour to about 10 hours. In an embodiment, the heat treatment may be performed during a time period in a range of, for example, about 1 hour to about 10 hours, about 3 hours to about 10 hours, about 5 hours to about 10 hours, about 0.1 hour to about 7 hours, about 0.1 hour to about 5 hours, about 0.1 hour to about 3 hours, about 1 hour to about 7 hours, about 3 hours to 7 hours, or about 1 hour to about 3 hours. In an embodiment, the size or density of the gold nanoclusters may be adjusted by performing the third calcination. In an embodiment, an exposed surface area toward the outside may be increased by having the gold nanoclusters protrude from the surface for about 0.1 hour to about 10 hours. In an embodiment, for example, the size or density of the gold nanoclusters may be increased.

The metal nanowire with the gold nanoclusters uniformly formed on the surface thereof at a high density may have an improved electron capture characteristic, improved electrical conductivity, and an improved optical characteristic including improved light absorption or optical emitting property, and thus the metal nanowire may be used in various electric devices.

Hereinafter, a preparation of an embodiment of metal nanowire including gold nanoclusters on the surface thereof will be described in greater detail.

In such an embodiment, the silicon nanowire including gold nanoclusters on the surface thereof were prepared.

In an embodiment, a gold thin film layer having a thickness in a range from 1.0 nm to 1.5 nm was vapor deposited by sputtering on a silicon substrate having a size of about 1.0×1.0 square centimeter (cm²) and a thickness of about 700 μm.

The silicon substrate, on which the gold thin film layer is formed, is disposed in a rapid thermal chemical vapor deposition (“RTCVD”) chamber including a halogen lamp, and then a calcination process is performed within the chamber in a hydrogen atmosphere at a pressure of about 0.5 Torr and a temperature of 700° C. for 10 minutes to form a gold-silicon island having a nano size in a range from about 50 nm to about 150 nm (called a “first calcination product”).

After the gold-silicon island is formed, a mixed gas of SiH₄ (about 2 sccm) and H₂ (about 50 sccm) is injected into the chamber while maintaining the pressure and temperature of the chamber at about 0.5 Torr and about 550° C., respectively, to grow a silicon nanowire, thereby preparing the silicon nanowire including gold nanoclusters on a surface thereof (called a “second calcination product”).

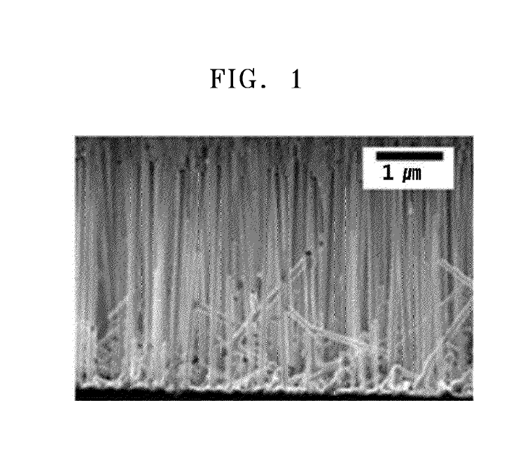

FIG. 1 is a scanning electron microscope (“SEM”) image of an embodiment of the silicon nanowire including the gold nanoclusters on the surface thereof, obtained by the method described above. As illustrated in FIG. 1, the silicon nanowire having a diameter in a range from about 30 nm to about 100 nm and a length in a range from about 0.5 μm to about 12 μm was obtained at a high density.

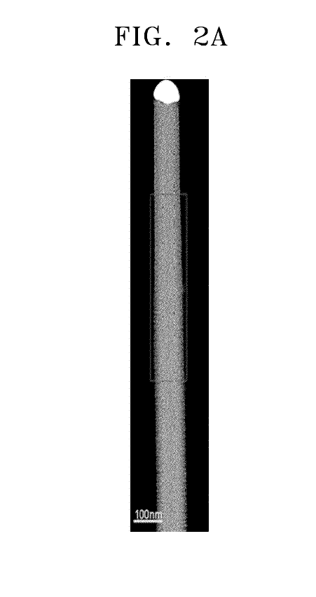

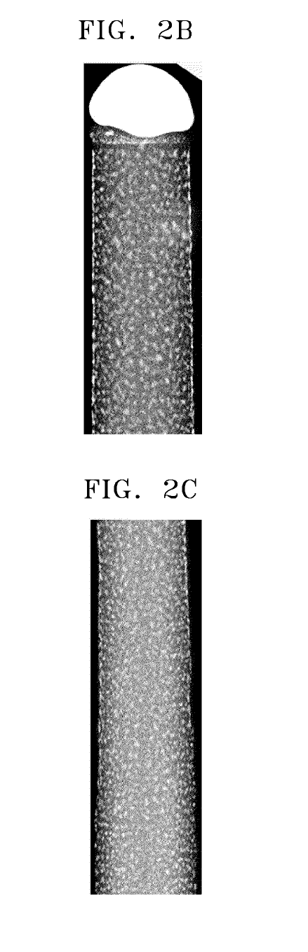

FIG. 2A is a Z-contrast image of an embodiment of a single silicon nanowire including the gold nanoclusters on the surface thereof at a high density. As shown in FIG. 2A, the silicon nanowire may have a uniform thickness.

FIG. 2B is a partial enlarged view of an upper part of the silicon nanowire including the gold nanoclusters at a high density on the surface thereof shown in FIG. 1. The gold nanoclusters each having a size in a range from about 2 nm to about 5 nm are uniformly distributed on the entire surface of the silicon nanowire, and a hemispherical cap of gold is provided on the upper part of the silicon nanowire.

FIG. 2C is a partial enlarged view of a central part of the silicon nanowire of FIG. 1, and the gold nanoclusters each having a size in a range from about 2 nm to about 5 nm are uniformly distributed on the entire surface of the silicon nanowire.

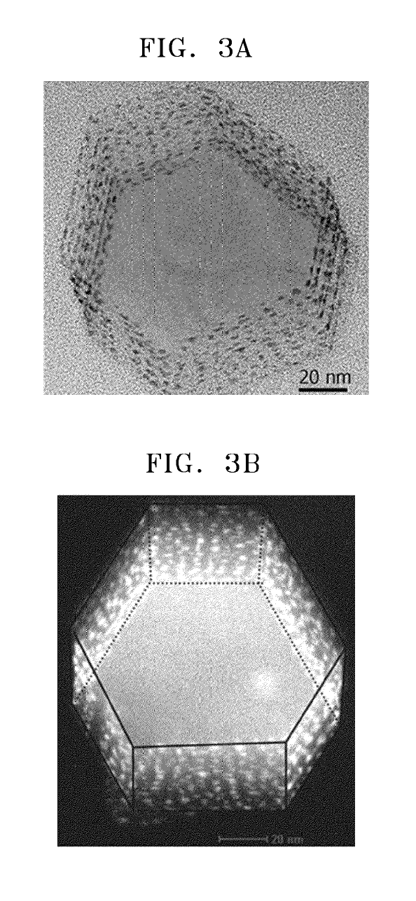

FIG. 3A is a transmission electron microscopy (“TEM”) image of a cross-section of an embodiment of the silicon nanowire of FIG. 1 including the gold nanoclusters on the surface thereof. FIG. 3B is a Z-contrast image showing a hexagon-shaped cross-section of the silicon nanowire including the gold nanoclusters uniformly distributed thereon at a predetermined interval.

In an embodiment, as shown in FIGS. 3A and 3B, the gold nanoclusters having a density of about 3.2×10¹² nanoclusters/cm² are provided on the surface of the silicon nanowire.

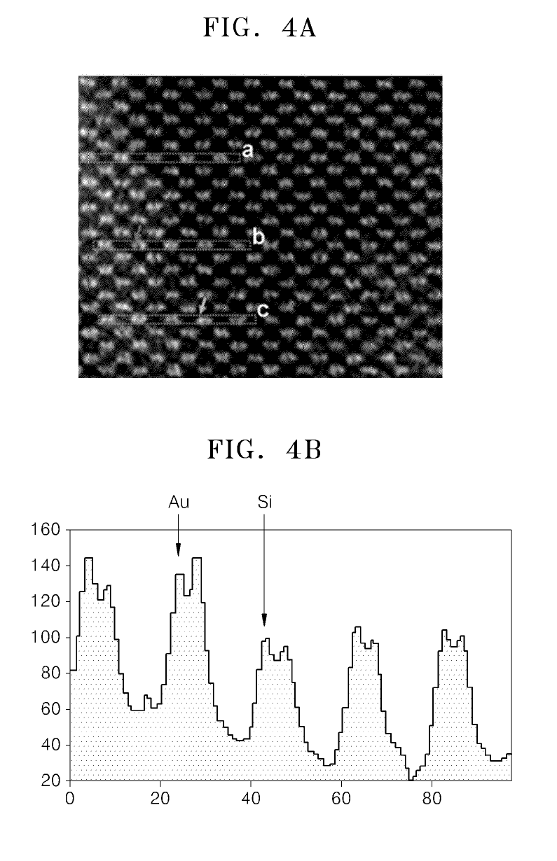

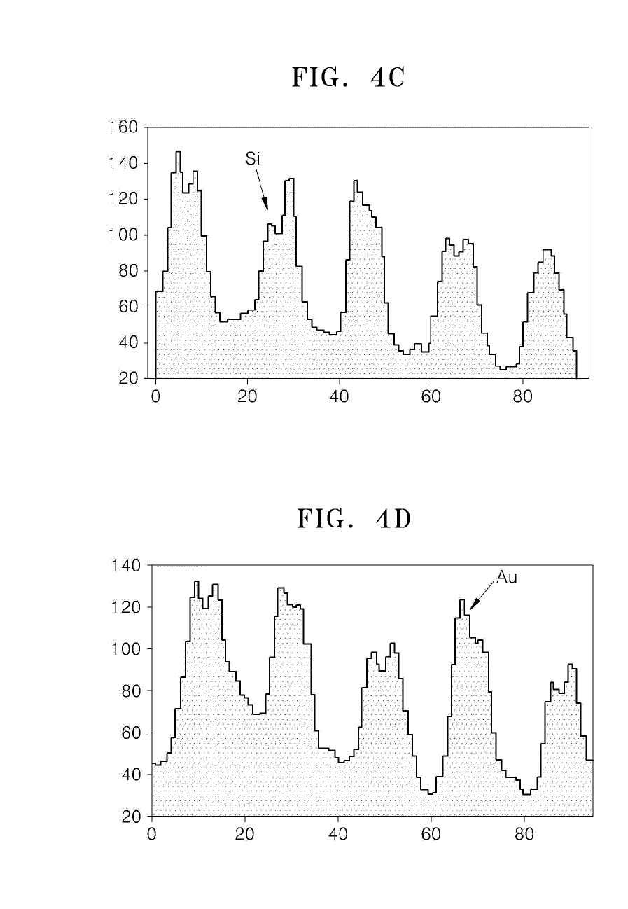

FIG. 4A is a high-resolution Z-contrast image showing a distribution of the gold nanoclusters on the surface of the silicon nanowire of FIG. 1. FIGS. 4B through 4D show distributions of contrast intensities of a, b, and c regions of FIG. 4A, respectively. The graphs of FIGS. 4B through 4D show that gold exists at a high intensity position, and silicon atoms exist at a low intensity position. Accordingly, FIG. 4A shows an over-saturated structure, in which gold atoms are substituted for silicon existing on the surface of the silicon nanowire.

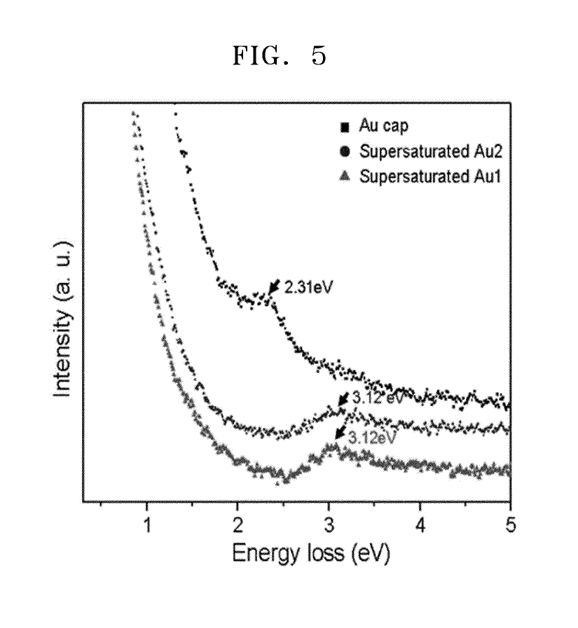

FIG. 5 is a graph showing a result of Monochrome-electron energy loss spectroscopy (“EELS”) data obtained by measuring an optical characteristic of a gold cap existing on the upper end portion of the silicon nanowire of FIG. 1 and an optical characteristic of the gold nanoclusters existing on the surface of the silicon nanowire. A surface plasmon resonance occurred at about 2.31 electron volts (eV) (about 537 nm) in the gold cap and occurred at about 3.12 eV (about 397 nm) in the gold nanoclusters.

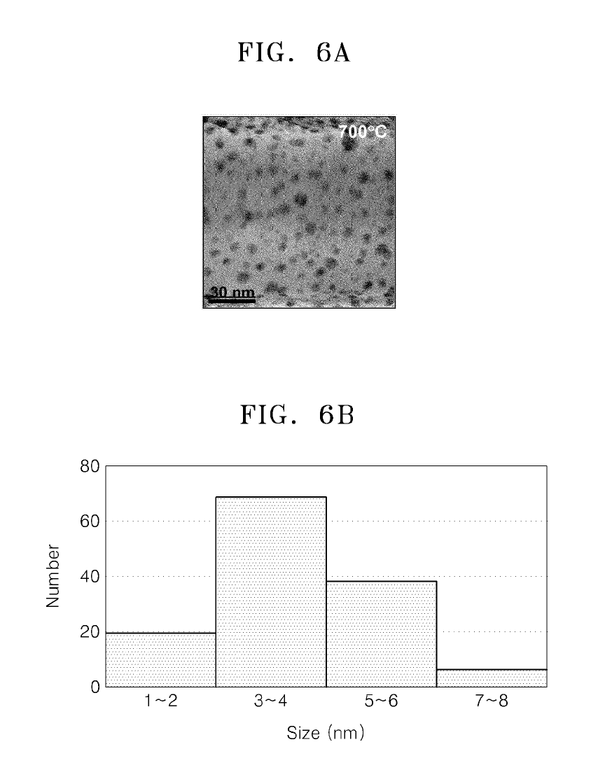

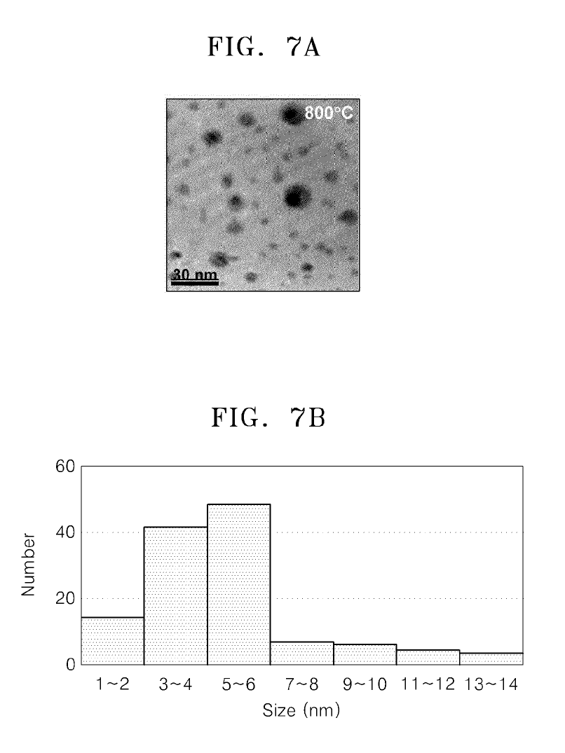

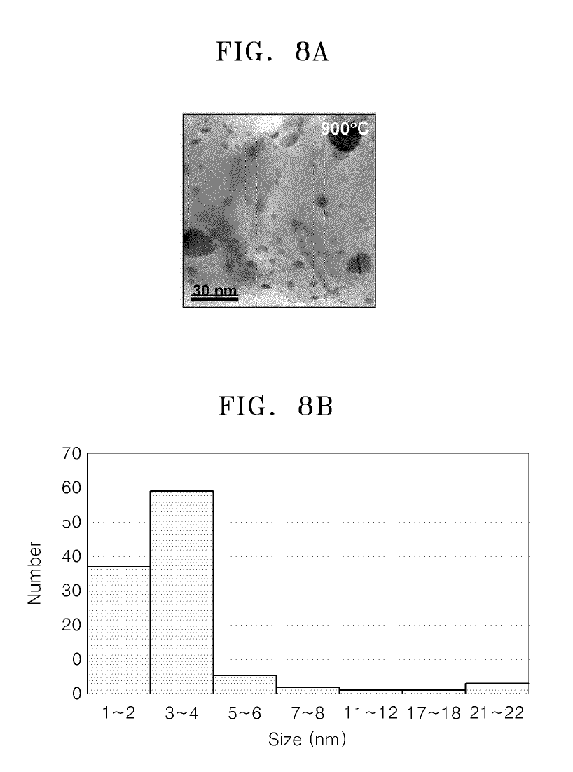

FIGS. 6A, 7A and 8A are partial enlarged views of the gold nanoclusters obtained by performing thermal treatments on silicon nanowires of FIG. 1 in a nitrogen atmosphere at temperatures of 700° C., 800° C. and 900° C., respectively, (hereafter, called a “third calcination product”), and FIGS. 6B, 7B and 8B are graphs showing distributions of the gold nanoclusters of FIGS. 6A, 7A and 8A according to the size thereof. The size of the gold nanoclusters may vary and be in a range from about 1 nm to about 30 nm according to a temperature of the thermal treatment. The third calcination may be performed, for example, at a temperature of about 300° C. through about 1,000° C. In an embodiment, an exposed surface area toward the outside may be increased by performing the third calcination, which makes the gold nanoclusters protrude from the surface of the silicon nanowire.

In such an embodiment, the size or density of the gold nanoclusters may be adjusted by performing the third calcination. In an embodiment, for example, an exposed area toward the outside may be increased by making the gold nanoclusters protrude from the surface of the silicon nanowire.

Hereinafter, an embodiment of separation of cells using metal nanowires with gold nanoclusters on the surface thereof will be described in greater detail.

In an embodiment, the nanowires may be prepared using silicon nanowires including gold nanoclusters on the surface thereof with a size in a range of about 3 nm to about 30 nm and a density of about 1×10⁶ gold nanoclusters/cm² to about 1×10¹⁶ gold nanoclusters/cm². In such an embodiment, the nanowire including the gold cluster may be prepared using the nanowire shown in FIG. 1 by performing the third calcination at a temperature of about 550° C.

In an embodiment, immobilization of anti-epithelial cell adhesion molecule (“anti-EpCAM”) antibodies on the nanowires is performed.

In such an embodiment, the silicon nanowires is exposed to about 310 nanograms per milliliter (ng/ml) of anti-EpCAM antibodies for about 20 minutes at a room temperature, e.g., in a range of about 15° C. to about 25° C. Then, the silicon nanowires are rinsed with a phosphate buffer saline (“PBS”) buffer 5 times such that nanowires having the anti-EpCAM antibodies bound with gold nanoclusters (hereinafter, “Au:SiNW2”) are prepared.

In an embodiment, the anti-EpCAM antibodies may be bound with the first calcination products described above using the same process (hereinafter, “Au:SiNW1”).

An exemplary experiment was performed on the separation of cells using metal nanowires with gold nanoclusters on the surface thereof. In such an experiment, silicon nanowires were prepared by rapid thermal chemical vapor deposition (“RTCVD”) as a control group. The silicon nanowires used were either nanowires having similar lengths and diameters of Au:SiNW1 or Au:SiNW2 or nanowires grown on a slant. The silicon nanowires were not modified with anti-EpCAM antibodies (hereinafter, ‘SiNW’).

In such an experiment, Au:SiNW1, Au:SiNW2 and SiNW were immobilized on a substrate. A material used to form the substrate was silicon.

In such an experiment, a gold plate was prepared as another control group (hereinafter, “AuP”). The AuP was not modified with anti-EpCAM antibodies. The AuP had a height, a width and a length of about 100 nm, about 10 mm and about 10 mm, respectively. The AuP was uniformly coated with gold.

EpCAM is a protein (reference sequence: NP_{—}002345) that in humans is encoded by the EPCAM gene. EpCAM has also been designated as tumor-associated calcium signal transducer 1 (“TACSTD1”) and cluster of differentiation 326 (“CD326”).

EpCAM is a pan-epithelial differentiation antigen that is expressed on almost all carcinomas. Its constitutional function is being elucidated. It is intricately linked with the Cadherin-Catenin pathway and hence the fundamental WNT pathway responsible for intracellular signaling and polarity. EpCAM is a carcinoma-associated antigen and is a member of a family that includes at least two type 1 membrane proteins.

In such an experiment, separations of cells using nanowires immobilized with anti-EpCAM antibodies were performed.

About 1,000 cells were placed on the nanowires (Au:SiNW1, Au:SiNW2 and SiNW) and the AuP by dropping about 10 microliters (μl) of sample including Michigan cancer foundation 7 (“MCF7”) cells onto the nanowires and the AuP. A cell concentration was about 10⁵ cells/ml. MCF7 is a breast cancer cell line isolated in 1970 from a 69-year-old Caucasian woman. The size of MCF7 cells is in a range of about 15 μm through about 30 μm.

Each of the nanowires and the cell sample mixture or the gold plate and the cell sample mixture were incubated for about 40 minutes in a CO₂ incubator maintaining about 5% CO₂ at about 37° C. Then, each of the nanowires (Au:SiNW1, Au:SiNW2 and SiNW) or the AuP was removed from the incubator at regular intervals of time to measure a number of cells attached on the surface. The number of cells was measured using a SEM or fluorescent microscopy method.

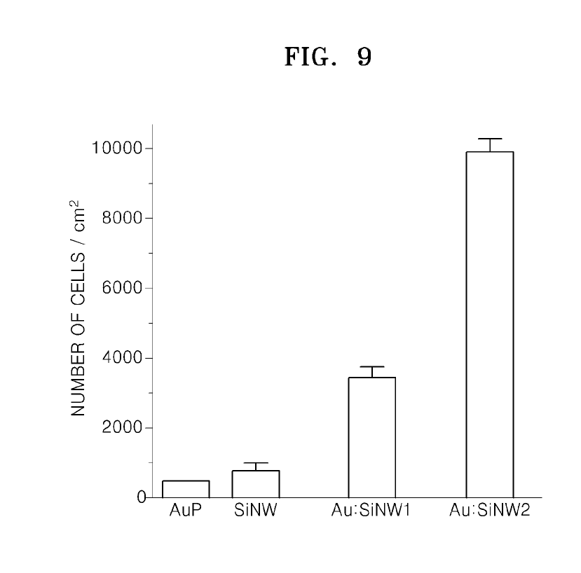

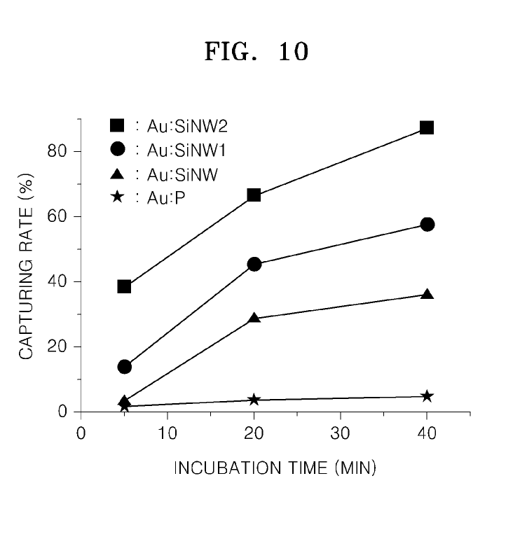

FIG. 9 is a graph showing the number of cells on the nanowires (Au:SiNW1, Au:SiNW2 and SiNW) and the AuP after about 5 minutes of incubation. As shown in FIG. 9, unexpectedly, Au:SiNW1 and Au:SiNW2 had significantly greater number of cells attached on than AuP and SiNW. As shown in FIG. 9, Au:SiNW2 showed a substantially higher cell-attaching efficiency compared to that of Au:SiNW1.

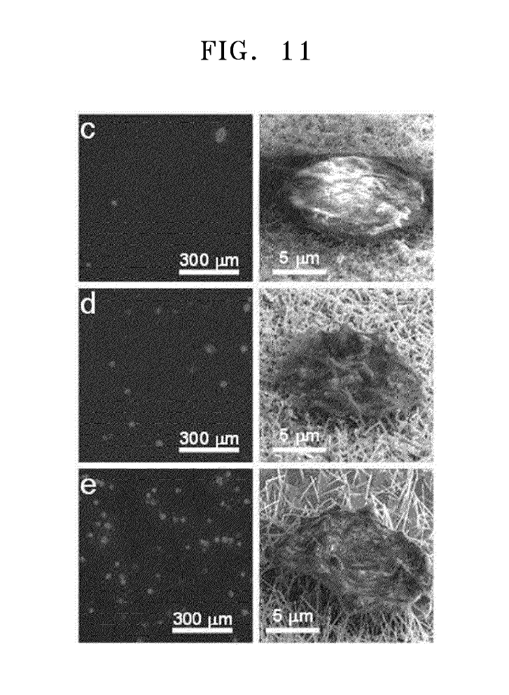

FIG. 10 is a graph showing the number of cells attached on the nanowires (Au:SiNW1, Au:SiNW2 and SiNW) or the AuP according to an incubation time. As shown in FIG. 10, Au:SiNW1 and Au:SiNW2 had substantially greater number of cells attached than AuP and SiNW. As shown in FIG. 10, Au:SiNW2 showed a substantially higher cell-attaching efficiency compared to that of Au:SiNW1.

FIG. 11 is an image showing the number of cells attached on Au:SiNW2 according to an incubation time. Images on the left side of FIG. 11 are 60 times magnified fluorescence microscope images and the images on the right side are 4000 times magnified scanning electron microscope images. In FIG. 11, C, D and E are images showing cells captured at about 5 minutes, about 20 minutes and about 40 minutes of incubation, respectively. As shown in FIG. 11, Au:SiNW2 has a high capture efficiency of about 80% or more at about 40 minutes of incubation.

In an embodiment, as shown in FIG. 11, regardless of a rough surface of Au:SiNW2, cells were attached on a large surface area with improved efficiency. In such an embodiment, a property of the surface of the gold nanoclusters and the nanowires may be changed according to the third calcination such that Au:SiNW2 has substantially improved number of cells attached than Au:SiNW1, but the embodiment is not limited to any particular mechanism.

In an embodiment, Au:SiNW1 or Au:SiNW2 attaching to the cells with a high efficiency is considered to be achieved by the gold clusters formed on the surface and moved to the surface of the nanowires according to the third calcination process.

According to embodiments of the invention described herein, a target material may bind to metal nanowires with a high affinity as each of the metal nanowires includes gold nanoclusters on the surface thereof.

Also, a target material in a sample may substantially efficiently bind to the metal nanowires using a method of binding the target material in the sample to the metal nanowires.

It should be understood that the exemplary embodiments described herein should be considered in a descriptive sense only and not for purposes of limitation. Descriptions of features within each of the embodiments should typically be considered as available for other similar features or aspects in other embodiments.

What is claimed is:

1. A metal nanowire comprising gold nanoclusters on a surface thereof, wherein the gold nanoclusters bind a target material to the metal nanowire.

2. The metal nanowire of claim 1, wherein the nanowire comprising the gold nanoclusters on the surface thereof exists in a form of a composition.

3. The metal nanowire of claim 1, wherein the gold nanoclusters have an average size in a range from about 1 nanometer to about 10 nanometers.

4. The metal nanowire of claim 1, wherein the gold nanoclusters are arranged on the surface of the metal nanowire in a density in a range from about 1×10⁶ nanoclusters per square centimeter to about 1×10¹⁶ nanoclusters per square centimeter.

5. The metal nanowire of claim 1, further comprising at least one metal selected from the group consisting of silicon (Si), nickel (Ni), iron (Fe), silver (Ag), aluminum (Al), germanium (Ge), gadolinium (Gd), copper (Cu), indium (In), titanium (Ti) and lead (Pb).

6. The metal nanowire of claim 5, wherein the at least one metal is silicon.

7. The metal nanowire of claim 1, wherein a diameter of the metal nanowire is in a range from about 10 nanometers to about 50 nanometers.

8. The metal nanowire of claim 1, wherein a length of the metal nanowire is in a range from about 0.5 micrometer to about 20 micrometers.

9. The metal nanowire of claim 1, wherein the target material comprises a molecule derived from an organism or a part thereof.

10. The metal nanowire of claim 1, wherein the target material is an antigen existing on a surface of a cell.

11. The metal nanowire of claim 1, wherein the target material is a circulating tumor cell.

12. The metal nanowire of claim 11, wherein a circulating tumor cell content in a medium is in a range of about 10⁻⁷ cells per milliliter to about 10⁻⁹ cells per milliliter.

13. A system for binding a target material, comprising:
a substrate; and
a plurality of metal nanowires immobilized on the substrate,
wherein each of the metal nanowires comprises gold nanoclusters on a surface thereof.

14. The system of claim 13, wherein the metal nanowires are vertically immobilized on the substrate.

15. The system of claim 13, wherein the metal nanowires are arranged on the substrate in a density in a range from about 1×10⁶ nanowires per square centimeter to about 1×10¹⁶ nanowires per square centimeter on the substrate.

16. A method of preparing a metal nanowire including gold nanoclusters on a surface thereof, the method comprising:
providing a gold thin layer on a metal substrate;
forming a gold-metal island by performing a first calcination process on the metal substrate including the gold thin layer thereon, in a chamber in a hydrogen atmosphere; and
performing a second calcination process on the metal substrate including the gold-metal island while injecting a mixed gas into the chamber, to grow the metal nanowire including gold nanoclusters on the surface thereof.

17. The method of claim 16, wherein the first calcination process is performed at a temperature in a range of about 300° C. to about 1,000° C. in a vacuum atmosphere in a range of about 0.1 torr to about 500 torrs.

18. The method of claim 17, wherein the second calcinations process is performed at a temperature in a range of about 500° C. to about 600° C. in a vacuum atmosphere in a range of about 0.1 torr to about 10 torrs.

19. The method of claim 18, wherein the mixed gas comprises SiH₄ and H₂.

20. The method of claim 16, further comprising:
adjusting a size or a density of the gold nanoclusters by performing a thermal treatment at a temperature in a range of about 300° C. to about 1,000° C. after the performing the second calcination process.

21. A method of binding a target material in a sample to a metal nanowire including gold nanoclusters on a surface thereof, the method comprising:
binding the target material to the metal nanowire by contacting the metal nanowire including the gold nanoclusters on the surface thereof to the sample including the target material.

22. The method of claim 21, further comprising:
separating the target material from the nanowire.

23. The method of claim 21, further comprising:
washing the nanowire after the binding the target material to the metal nanowire.

24. The method of claim 21, wherein further comprising:
lysing the target material by irradiating a laser on the nanowire, after the binding the target material to the metal nanowire.

25. The method of claim 22, wherein further comprising:
lysing the target material by irradiating a laser on the nanowire, after the separating the target material from the nanowire.

26. The method of claim 21, further comprising:
preparing the metal nanowire including gold nanoclusters on the surface thereof,
wherein the preparing the metal nanowire including gold nanoclusters on the surface thereof comprises:
providing a gold thin layer on a metal substrate;
forming a gold-metal island by performing a first calcination process on the metal substrate including the gold thin layer thereon, in a chamber in a hydrogen atmosphere; and
performing a second calcination process on the metal substrate including the gold-metal island while injecting a mixed gas into the chamber to grow the metal nanowire including the gold nanoclusters on the surface thereof.

27. The method of claim 26, wherein the first calcination is performed at a temperature in a range of about 300° C. to about 1,000° C. in a vacuum atmosphere in a range of about 0.1 torr to about 500 torrs.

28. The method of claim 26, wherein the second calcination is performed at a temperature in a range of about 500° C. to about 600° C. in a vacuum atmosphere in a range of about 0.1 torr to about 10 torrs.

29. The method of claim 26, wherein the mixed gas comprises SiH₄ and H₂.

30. The method of claim 26, further comprising:
adjusting a size or a density of the gold nanoclusters by performing a thermal treatment at a temperature in a range of about 300° C. to about 1,000° C. after the performing the second calcination process.
